SEQUENCE LISTING (2 sequences)

SEQ ID NO: 1 - protein, 52 aa, Artificial Sequence
  Synthetic peptide
GPLGSPGIRGSCVNPNTGKLIQGAPTIRGDPECHLFYNEQQEACGVHTQRMT

SEQ ID NO: 2 - protein, 41 aa, Artificial Sequence
  Synthetic peptide
CVNPNTGKLIQGAPTIRGDPECHLFYNEQQEACGVHTQRMT

(19) United States
(12) Patent Application Publication
(10) Pub. No.: US 2020/0206313 A1
(43) Pub. Date: Jul. 2, 2020

(54) POLYPEPTIDE NANOTUBES LINKED BY DISULFIDE BONDS FOR TARGETED DELIVERY OF CYTOTOXIC THERAPIES

(71) Applicants: MUSC FOUNDATION FOR RESEARCH DEVELOPMENT, Charleston, SC (US); INDIAN INSTITUTE OF SCIENCE, Bangalore (IN)

(72) Inventors: Steven A. ROSENZWEIG, Mt. Pleasant, SC (US); Hanudatta S. ATREYA, Bangalore (IN); Asampilli GITANJALI, Bangalore (IN); Paturu KONDAIAH, Bangalore (IN)

(73) Assignees: MUSC FOUNDATION FOR RESEARCH DEVELOPMENT, Charleston, SC (US); INDIAN INSTITUTE OF SCIENCE, Bangalore, Karnataka, IN (US)

(21) Appl. No.: 16/724,436

(22) Filed: Dec. 23, 2019

(30) Foreign Application Priority Data
Dec. 26, 2018 (IN) 201811049170

(51) Int. Cl. A61K 38/17; A61P 35/00

(52) U.S. Cl. CPC A61K 38/1777; A61P 35/00

(57) ABSTRACT

Provided herein is a self-assembling polypeptide-based nanotube system having the ability to target cancer cells. The nanotubes target the cancer cell surface through integrin engagement with the help of multiple RGD units present along their surface and release their drug payload in a sustained manner. In addition, the nanotubes can be utilized for cellular imaging using any covalently tagged fluorescent dye. Provided herein is a self-assembling polypeptide-based nanotube system having the ability to target cancer cells. The nanotubes target the cancer cell surface through integrin engagement with the help of multiple RGD units present along their surface and release their drug payload in a sustained manner. In addition, the nanotubes can be utilized for cellular imaging using any covalently tagged fluorescent dye.

The invention was made with government support under Grant Nos. RO1 CA78887, R01CA134845, P30 CA138313, 5F30-DE015249 and T32-HL0726 awarded by the National Institutes of Health and under Grant No. N6311601MD10004 awarded by the Department of Defense. The government has certain rights in the invention.

This application a Paris Convention filing based on India Patent Application No. 201811049170, filed Dec. 26, 2018, the entirety of which is incorporated herein by reference.

Pursuant to 37 C.F.R. 1.821(c), a sequence listing is submitted herewith as an ASCII compliant text file named “MESCP0114US_ST25.txt”, created on Dec. 17, 2019 and having a size of ˜2 kilobytes. The content of the aforementioned file is hereby incorporated by reference in its entirety.

### BACKGROUND OF THE INVENTION

### 1. Field of the Invention

The present invention relates generally to the field of molecular biology and oncology. More particularly, it concerns structured polypeptide delivery vehicles for therapeutic agents.

### 2. Description of Related Art

The development of multimodal systems combining imaging and drug delivery components have come into focus due to their theranostic efficacy (Alibolandi et al., 2016). Different strategies for designing nanocarriers have been proposed such as nanogels, polymeric micelles, liposomes with various targeting agents (Cuggino et al., 2016; Wei et al., 2015; Tomitaka et al., 2018; Poshteh Shirani et al., 2017). A desirable property of such systems is the ability to target the tumor cells and release the drug stably in the blood stream (Wei et al., 2015). To achieve this, targeted delivery systems have been proposed (Wang and Thanou, 2010). The Arg-Gly-Asp (RGD) is the most widely studied peptide for specific targeting in the biomaterials field (Bellis, 2011). This tripeptide has proved to be very effective in binding integrin receptors as efficiently as the principal integrin-binding domains within ECM proteins such as fibronectin, vitronectin and fibrinogen (Arnaout et al., 2005). Integrins are heterodimeric cell surface receptors with alpha and beta subunits (Arnaout et al., 2005). They mediate interaction among cells via their adhesion to the extracellular matrix. There are 24 integrin heterodimers, and several of them are upregulated in various tumor types including breast cancer (Desgrosellier et al., 2010). The RGD motif exhibits association with several types of integrins such as α5β1, αvβ3 and αvβ5 integrins (Hynes et al., 2002).

In recent studies, nanotubular structures have been formed by self-assembly of a polypeptide fragment at the C-terminal end (residues 249-289) of human insulin-like growth factor binding protein-2 (hIGFBP-2₂₄₉₋₂₈₉) (Swain et al., 2010). Wild type hIGFBP-2₂₄₉₋₂₈₉ has two cysteines in its primary sequence. However, the polypeptide fragment considered had an additional cysteine due to a mutation at R281. The polypeptide (hIGFBP-2₂₄₉₋₂₈₉ (R281C)) thus had an odd number of cysteines, which resulted in spontaneous self-assembly to form soluble nanotubular structures via intermolecular disulfide bonds. Further, the polypeptide fragment contains a RGD motif in its sequence. Upon formation of nanotubes, an array of RGD is displayed on the surface providing a unique feature for active targeting of cancer cells through integrin binding. Therefore, cellular imaging and drug delivery using these nanotubes is an attractive option for targeting cancer cells.

### SUMMARY OF THE INVENTION

In a first embodiment, the present disclosure provides therapeutic compositions comprising a polypeptide tubule composed of polypeptide subunits that are linked by cysteine disulfide bonds and a therapeutic molecule encapsulated in said polypeptide tubule.

In certain aspects, the polypeptide subunits each have identical sequences. The polypeptide subunits may be 10-100, 10-80 or 25-60 amino acids in length, such as 25, 30, 35, 36, 37, 38, 39, 40, 41, 42, 43, 44, 45, 46, 47, 48, 49, 50, 51, 52, 53, 54, 55, 56, 57, 58, 59, or 60 amino acids. In further aspects, the polypeptide subunits are 10-25 amino acids in length. In some aspects, the polypeptide subunits comprise an RGD motif. In certain aspects, the polypeptide tubule can bind to cell surface integrin.

In some aspects, the polypeptide subunits each comprise at least 3 cysteine positions. The polypeptide subunits may comprise an intramolecular disulfide bond between a first and second cysteine of the same subunit and an intermolecular bond between a third cysteine and a cysteine from a different subunit. In particular aspects, the polypeptide subunits comprise an amino acid sequence at least 80% (e.g., at least 81%, 82%, 83%, 84%, 85%, 86%, 8′7%, 88%, 89%, 90%, 91%, 92%, 93%, 94%, 95%, 96%, 97%, 98% or 99%) identical to CVNPNTGKLIQGAPTIRGDPECHLFYNEQQEACGVHTQRMT (SEQ ID NO: 2) or GPLGSPGIRGSCVNPNTGKLIQGAPTIRGDPECHLFYNEQQEACGVHTQRMT (SEQ ID NO: 1). In certain aspects, the polypeptide subunits comprise an amino acid sequence at least 85%, 86%, 87%, 88%, 89%, 90%, 91%, 92%, 93%, 94%, 95%, 96%, 97%, 98% or 99% identical to SEQ ID NO: 2. In specific aspects, the polypeptide subunits comprise an amino acid sequence identical to SEQ ID NO: 2. In some aspects, the polypeptide subunits comprise an amino acid sequence at least 85%, 86%, 87%, 88%, 89%, 90%, 91%, 92%, 93%, 94%, 95%, 96%, 97%, 98% or 99% identical to SEQ ID NO: 1. In particular aspects, the polypeptide subunits comprise an amino acid sequence identical to SEQ ID NO: 1. In some aspects, the polypeptide tubule is PEGylated.

In some aspects, the therapeutic molecule is a cytotoxic agent. In certain aspects, the therapeutic molecule is a chemotherapy, radiotherapy, gene therapy, surgery, hormonal therapy, anti-angiogenic therapy or cytokine therapy agent. In particular aspects, the therapeutic molecule is a chemotherapeutic agent. For example, the therapeutic molecule is Doxorubicin.

In another embodiment, there is provided a method of treating a subject in need thereof comprising administering an effective amount of a composition of the embodiments (e.g., composition comprising a polypeptide tubule composed of polypeptide subunits that are linked by cysteine disulfide bonds and a therapeutic molecule encapsulated in said polypeptide tubule).

In some aspects, the therapeutic molecule is a chemotherapeutic agent and the wherein the subject has a cancer. The cancer may be oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

In some aspects, the inhibitor of composition is administered daily. In certain aspects, the composition is administered on a continuous basis.

In additional aspects, the method further comprises administering an additional anti-cancer therapy. In some aspects, the additional anti-cancer therapy is chemotherapy, radiotherapy, gene therapy, surgery, hormonal therapy, anti-angiogenic therapy or immunotherapy. In certain aspects, the composition is administered intravenously, subcutaneously, intraosseously, orally, transdermally, in sustained release, in controlled release, in delayed release, as a suppository, or sublingually. In particular aspects, administering composition comprises local, regional or systemic administration. In some aspects, the composition is administered two or more times.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1a-d: Oligomerization through self-assembly of hIGFBP-2₂₄₉₋₂₈₉ (R281C). (FIG. 1a) SDS-PAGE profile of the oligomerization M: marker, A: Monomer, B: Dimer (day 1), C: Trimer (day 2), D: multiple oligomer species (day 4); (FIG. 1b) 2D [¹⁵N, ¹H] heteronuclear single quantum correlation (HSQC) spectra on day 1 and day 4, purple color dotted line circles depict the pairs of peaks representing old and new species for corresponding amino acid residues; (FIG. 1c) Cys44 was monitored for oxidation of —SH to disulfide (—SS—) with oligomerization, based on ¹³C^{β} shifts observed in the 2D [¹³C-¹H] HSQC NMR experiment, (FIG. 1d) TEM images of the hIGFBP-2₂₄₉₋₂₈₉ nanotubes.

FIGS. 2a-d: Fluorescence emission spectra of monomers and nanotubes. (FIG. 2a) Fluorescence emission spectra of monomer (red) and nanotube (black). (FIG. 2b) Normalized fluorescence emission spectra of hIGFBP-2₂₄₉₋₂₈₉ nanotubes at three different pH: pH 2 (green), pH 7 (black), pH 12 (red). All spectra were acquired with similar concentrations of nanotube at 298 K. (FIG. 2c) Fluorescence emission spectra of hIGFBP-2₂₄₉₋₂₈₉ nanotubes acquired at different temperatures. (FIG. 2d) Fluorescence intensity (λₘₐₓ) at different temperatures for nanotubes at pH 2 (blue) and pH 12 (red).

FIGS. 3a-b: (a) AFM image of the nanotubes, (b) Line profile used for calculating the averaged diameter.

FIG. 4: Western blot showing integrin specificity. C: Untreated as control, C+: Control in presence of an inhibitor, N: Nanotube, N+: Nanotube in presence of an Inhibitor, Inhibitor—RGDS, 1 h treatment followed by 1 h treatment of samples.

FIG. 5: Cancer cell imaging with nanotube-FITC. Cellular interaction of nanotube-FITC (top row) and free FITC (bottom row) to HeLa cells at 4 h (left column), Cellular binding of nanotube-FITC and free FITC following 1 h treatment of the integrin inhibitor given after 4 h of incubation of the conjugate with the cells (right column). Cell nuclei are stained with DAPI (blue color).

FIGS. 6a-b: In vitro drug release in response to pH and Redox stimuli (FIG. 6a) pH triggered release of DOX in release medium from NTDOX in comparison to Free DOX release at physiological pH (FIG. 6b) pH and redox responsive release of DOX from NTDOX at pH 5 and 7.4.

FIG. 7a-b: Cytotoxicity evaluation using MTT assay. (FIG. 7a) HeLa (FIG. 7b) MDAMB231. The IC₅₀ of nanotube-DOX for HeLa is 0.4 μg/ml and free DOX is 0.45 μg/ml. In the case of MDAMB231, IC₅₀ of Nanotube-DOX is 2 μg/ml and free DOX is 2.3 μg/ml. suggesting the nanotube-DOX matches the efficiency of DOX with minimal cytotoxicity.

FIGS. 8a-c: Cellular uptake of DOX. (FIG. 8a) Percentage of DOX internalized cells at different time points as analyzed using FACS. (FIG. 8b) and (FIG. 8c) Histogram plots comparing percentage of DOX internalized in HeLa and HaCaT cells, respectively, for nanotube-DOX, free DOX and untreated cells (control). The FACS data for HaCaT cells is shown in FIG. 15.

FIG. 9: Cellular uptake of DOX from nanotube-DOX by HeLa at two time points.

FIG. 10: Scheme of self-assembling nanotube formation and targeting of cancer cells.

FIG. 11: Monitoring oligomerisation as a function of time using 2D [15N, 1H] HSQC NMR experiment on day 1, day 2 and day 4. HSQC spectra (Top), expanded region representing the new species arising as a function of time (bottom).

FIGS. 12a-d: Size distribution and Zeta potential profiles. (FIG. 12a) Dynamic light scattering profile of nanotubes; Zeta potential for the (FIG. 12b) nanotube and (FIG. 12c) nanotube-DOX system; (FIG. 12d) Comparison of zeta potentials for nanotube and nanotube-DOX.

FIGS. 13a-c: Nanotube-FITC preparation and characterization. (FIG. 13a) UV-Vis spectra for free FITC and Nanotubes Conjugated with FITC (FIG. 13b) Upon UV excitation FITC tagged nanotubular intermediate (FIG. 13c) Cellular uptake of nanotube-FITC in HeLa cells at 8 h.

FIG. 14: Free-FITC interaction with HeLa cells. Free FITC rapidly diffused in the cells in absence of the inhibitor (left column) and cell uptake following 1 h treatment of the integrin inhibitor given after 4 h of incubation of the conjugate with the cells (right column). Cell nuclei are stained with DAPI (blue color) and shown are the overlay of images (merged) acquired using FITC and DAPI channel.

FIGS. 15a-c: Nanotube-DOX preparation and characterization. (FIG. 15a) TEM image after loading Doxorubicin on nanotubes (FIG. 15b) Confocal image of Doxorubicin loaded Nanotube coated on a glass surface, (FIG. 15c) UV-vis spectrum showing characteristic peak for Doxorubicin.

FIGS. 16a-b: In vitro drug release profile of nanotube-DOX. (FIG. 16a) at physiological pH. (FIG. 16b) at acidic pH.

FIG. 17: Cytotoxicity assay. Comparing cytotoxicity profile of nanotubes obtained in three different cell lines at 48 h.

FIG. 18: Cellular uptake of DOX via nanotube-DOX, free DOX and untreated (Control) for HaCaT cells. Percentage of cell population positive for internalized DOX fluorescence at different time points.

### DESCRIPTION OF ILLUSTRATIVE EMBODIMENTS

Specificity, efficient accumulation and drug release constitute challenging hurdles for cancer therapy. Provided herein is a self-assembling polypeptide-based nanotube system having the ability to target cancer cells. The nanotubes target the cancer cell surface through integrin engagement with the help of multiple RGD units present along their surface and release their drug payload in a sustained manner. In addition, the nanotubes can be utilized for cellular imaging using any covalently tagged fluorescent dye. They are stable over a wide range of temperature and pH due to intermolecular disulfide bonds formed during the self-assembly process. Taken together this system opens up new avenues for multimodal formulation in cancer therapy.

### I. THERAPEUTIC AGENTS

In some aspects, therapeutic agents for use according to the embodiments include chemotherapeutic agents. For example, in some aspects the chemotherapeutic agent is a protein kinase inhibitor such as a EGFR, VEGFR, AKT, Erb 1, Erb2, ErbB, Syk, Bcr-Abl, JAK, Src, GSK-3, PI3K, Ras, Raf, MAPK, MAPKK, mTOR, c-Kit, eph receptor or BRAF inhibitors. Nonlimiting examples of protein kinase inhibitors include Afatinib, Axitinib, Bevacizumab, Bosutinib, Cetuximab, Crizotinib, Dasatinib, Erlotinib, Fostamatinib, Gefitinib, Imatinib, Lapatinib, Lenvatinib, Mubritinib, Nilotinib, Panitumumab, Pazopanib, Pegaptanib, Ranibizumab, Ruxolitinib, Saracatinib, Sorafenib, Sunitinib, Trastuzumab, Vandetanib, AP23451, Vemurafenib, MK-2206, GSK690693, A-443654, VQD-002, Miltefosine, Perifosine, CAL101, PX-866, LY294002, rapamycin, temsirolimus, everolimus, ridaforolimus, Alvocidib, Genistein, Selumetinib, AZD-6244, Vatalanib, P1446A-05, AG-024322, ZD1839, P276-00, GW572016 or a mixture thereof.

Yet further chemotherapeutic agents include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein tansferase inhibitors, transplatinum, and pharmaceutically acceptable salts, acids or derivatives of any of the above.

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Preparation of the Protein Nanotubes.

Expression and purification of monomer: To express the monomeric form of the protein, the pGEX-6p2-IGFBP-2₂₄₉₋₂₈₉ (C281) plasmid (Swain et al., 2010) was used for transformation of BL21 (DE3) pLysE E. coli cells. The Glutathione-S-Transferase (GST) Gene Fusion system was used for the over expression and purification of the construct (Kibbey et al., 2006). The amino acid sequence of the polypeptide is SEQ ID NO: 1: GPLGSPGIRGSCVNPNTGKLIQGAPTIRGDPECHLFYNEQQEACGVHTQRMT (SEQ ID NO: 1). The underlined first 11 N-terminal residues correspond to additional amino acids introduced by the gene fusion system as described previously (Kibbey et al., 2006). For bacterial culture, 10 ml of Luria-Bertani (LB) (Hi-media, M575) medium containing 100 μg/ml Ampicillin (Himedia, CMS645) was inoculated with a transformed colony and grown overnight at 37° C. Later, cells were regrown to mid-log phase (O.D_{600 nm}˜0.6) at 37° C. with 100-fold dilution from overnight primary culture in fresh 100 μg/ml Ampicillin containing LB medium. Overexpression of the fusion protein was achieved by inducing the cells with 1 mM isopropyl β-D-thiogalactoside (IPTG, Calbiochem, 420322, India) for 6 h at 30° C. Cells were harvested by centrifugation at 6000 rpm for 20 min, followed by cell lysis in phosphate buffered saline (PBS), pH 7.5, 1 mM PMSF (phenylmethylsulfonyl fluoride, Himedia, cat no. India), on ice by sonication in six steps, 20 s cycles each, with an intervening period of 2.0 min. Cell lysate was centrifuged (twice) at 30000 g for 45 min at 4° C. to separate insoluble cell debris and the soluble fraction containing the protein. Soluble fraction was loaded on 50% slurry of pre-equilibrated Glutathione-Sepharose beads (Novagen, 70541, India) with PBS at 4° C., for 3 h on nutator. The fusion protein-bound to the affinity beads was collected by centrifugation at 4000 rpm for 5 min and washed three times with 10 bed volumes of PBS (pH 7.5, 50 mM phosphate, 50 mM NaCl). It was further washed three times with 10 bed volumes of high (25 mM HEPES, 0.05% NaN₃, 0.5 M NaCl, and 0.1% Triton X-100, pH 7.5) and low (25 mM HEPES, 0.05% NaN₃, 0.1 M NaCl, and 0.1% TritonX-100, pH 7.5) salts. This was followed by washing twice with cleavage buffer (1.5 M NaCl, 0.5 M Tris-HCl, pH 7.5) to remove impurities and nonspecifically bound protein. On column cleavage was performed by pre-scission protease HRV-3C Protease (Human rhinovirus 3C protease cleaves at Q-G bonds, Millipore, 71493-3, India). 10 μl (20 units) of HRV-3C Protease was added for each milliliter of glutathione-Sepharose bed volume, in addition with 490 μl of cleavage buffer. It was then nutated at 4° C. for 16 hrs. Cleaved protein was eluted by centrifuging at 4000 rpm, 5 min and further subjected to exchange with PBS buffer (pH 7.4). Purity of purified protein was confirmed by loading it on SDS-PAGE.

The monomeric form of the protein was then allowed to undergo oligomerization in PBS to form self-assembled nanotubes. No external agent was necessary for initiating the oligomerization. Formation of the nanotubes was monitored by SDS-PAGE, NMR spectroscopy, TEM and Dynamic Light Scattering (DLS).

Characterization of Nanotubes by NMR Spectroscopy.

The monomer (hIGFBP-2₂₄₉₋₂₈₉ (Cys²⁸¹)) (SEQ ID NO: 1) and the nanotubes formed were characterized by NMR spectroscopy. The incorporation of NMR active isotopes i.e. ¹³C and ¹⁵N in to the protein was achieved by growing the transformed BL21 (DE3) pLysE E. coli cells in a minimal (M9) medium containing ¹³C₆-glucose and/or ¹⁵NH₄Cl as the sole sources of anabolic carbon and nitrogen (Atreya, 2012; Muchmore et al., 1989). Bacterial culture and purification of the proteins were carried out using previously described protocol. Protein estimation was done using Lowry's method (Lowry et al., 1951).

NMR Experiments.

About 0.8 mM purified protein was dissolved in PBS and 5% ²H₂O (for locking). All NMR data were recorded at 298 K on a Bruker Avance III 800 MHz NMR spectrometer equipped with a cryogenically cooled triple resonance probe. Two-dimensional (2D) [¹³C, ¹H] HSQC was acquired with 8 transients and 256 complex points with a measurement time of 40 min each and 2D [¹⁵N, ¹H] HSQC spectra were acquired with 2 transients and 256 complex points, with a measurement time of 10 min each. The 2D [¹⁵N, ¹H] HSQCs experiment was used for time dependent monitoring of oligomerization of self-assembling hIGFBP-2₂₄₉₋₂₈₉ (Cys²⁸¹) for 3 days and 2D [¹³C, ¹H] HSQCs were acquired to monitor the oxidation of Cysteine during self-assembly as a function of time for 8 days.

Assessment of Oligomerization by SDS-PAGE.

Self-assembly of hIGFBP-2₂₄₉₋₂₈₉ (Cys²⁸¹) into nanotubular structures in the initial stages was monitored by SDS-PAGE. This was carried out in parallel with the 2D [¹⁵N, ¹H] HSQC NMR experiment mentioned above. Protein fractions were collected on day 1, day 2 and day 3 and stored at −20° C. to arrest the oligomerization. Samples for SDS-PAGE were prepared in a buffer containing 240 mM Tris-HCl (pH 6.8), 8% (w/v) SDS, 0.1% Bromophenol blue and 40% (v/v) glycerol. Protein fractions taken in this buffer were heated at 96° C. for 10 min, and then centrifuged briefly. The samples were resolved on 15% SDS-polyacrylamide gels in Tris-glycine electrophoresis buffer (pH 8.3) containing 25 mM Tris base, 250 mM glycine and 0.1% SDS at 100 Volts for 120 min. Coomassie brilliant blue g-250 staining was performed for visualization of oligomeric bands.

Preparation of Nanotube-DOX.

Self-assembled nanotubes were subjected to exchange with Milli-Q water using a 3 kDa 15 ml Centricon tube (Millipore) spun at 4000 rpm at 4° C. Stability of the nanotube in pure water was confirmed by TEM. The sample was lyophilized and the dry weight was taken to quantify the yield. Adsorption of Doxorubicin hydrochloride (DOX) (purchased from Sigma, India) on nanotube was carried out by mixing a sample of DOX prepared in water at a concentration of 2 mM with 100 μM of nanotubes (corresponding to a protein: DOX ratio of 1:20) and the mixture was nutated for 12 h at 4° C. Unbound DOX was removed by dialysis against pure water using 1 kDa membrane (Millipore). Drug Encapsulation efficiency and drug loading content was calculated as below:

Encapsulation efficiency (EE) (%)=(W₀−W_{d}/W₀)×100

Loading content (LC) (%)=(W₀−W_{d}/W_{NT})×100

W₀ represents the initial amount of drug fed, W_{d} represents amount of drug released in dialysate, W_{NT} represents weight of Nanotubes used for drug loading.

Release of DOX from the Nanotube.

In-vitro drug release experiments were conducted using the dialysis method. The Nanotube-DOX conjugate was dialyzed against medium under different conditions: pH 5, pH 7.4, pH 7.4 with addition of 10 mM GSH, pH 5+10 mM GSH, pH 7.5+10 mM GSH+Enzyme (trypsin) and pH 5+10 mM GSH+trypsin. GSH was used to stimulate redox conditions and trypsin was used to investigate the effect of a non-specific enzyme on nanotube drug carrying ability. As a control free DOX was dialyzed against the same medium at physiological pH (7.4). All dialysis experiments were performed using a dialysis kit having a molecular mass cutoff of 1 kDa. Samples were dialyzed against the DMEM medium without phenol red (Thermofisher scientific) containing 1% P/S (Penicillin-Streptomycin, Cat no, India), 10% FBS (Fetal bovine serum, RM10432, Himedia, India) separately, with gentle and constant stirring at 37° C. At specific time points, aliquots of the medium were collected and absorbance at 495 nm was measured to quantify the release of DOX.

Preparation of Nanotube Conjugated with FITC.

Fluorescein isothiocyanate (FITC) (Sigma, India) was conjugated to nanotubes using the procedure described previously (Goding et al., 1976). All steps were performed in dark conditions to avoid photo hydrolysis of FITC. First, a solution containing 1 mg/ml of the protein nanotubes was prepared in borate buffer (0.5 M, pH 8.5). The FITC was added to this solution in the ratio of 1:10 (nanotube: FITC) with continuous stirring. Reaction mixture was shaken well to obtain uniform dissolution of FITC and protein. It was then kept at 4° C. for nutation overnight. The product (nanotube-FITC conjugate) was subjected to dialysis against borate buffer to remove unconjugated FITC using a 1 kDa membrane cut-off eventually exchanging with Milli-Q water. After complete buffer exchange, the final conjugate was lyophilized and quantified. For experimental use, the lyophilized conjugate was dissolved and stored in PBS (50 mM, pH 7.4) with 0.02% NaN₃ (an antibacterial agent). FITC/Protein (F/P) ratio of the conjugate was calculated according to the equations

$$Molar F \text{/} P = \frac{MW}{389} \times \frac{{A}_{495} \text{/} 195}{( {A}_{280} - ( 0.35 \times {A}_{495} ) ) \text{/} {E}^{0.1 \%}} = {A}_{495} \times C$$

$$\text{Where} , C = \frac{MW \times {E}_{280}^{0.1 \%}}{389 \times 195}$$

C is a constant value given for a protein, MW is the molecular weight of the protein, 389 Da is the molecular weight of FITC, 195 is the absorption E0.1% of bound FITC at 490 nm at pH 8.0 (0.35×A495) is the correction factor due to the absorbance of FITC at 280 nm³⁴, E 0.1% is the absorption at 280 nm of a protein at 1.0 mg/ml.

Transmission Electron Microscopy (TEM).

For acquiring TEM images, the nanotubes were prepared at room temperature (25° C.) by allowing self-assembly under redox control i.e. having no reducing agent present in PBS buffer. The TEM studies were recorded under 200 kV on a Technai F-30 TEM instrument equipped with a cryoprobe. Drop casting method was used and samples were stained with Uranyl acetate (0.5%), then air dried for 1 h and desiccated for overnight on copper grids of 200 mesh.

Fluorescence Spectroscopy.

Fluorescence spectra were acquired on a Perkin Elmer Fluorescence spectrometer. Spectra were recorded for protein samples prepared in 10 mM Tris buffer at pH 7 or 12 (i.e., neutral and basic pH) and 10 mM HEPES buffer at pH 2 (acidic pH) and data were recorded using a 1.0 cm path length quartz cuvette. An excitation wavelength of 280 nm (tyrosine excitation) was used having slit width 2.5 nm and emission spectra were collected from 300 nm to 550 nm with λₘₐₓ at 340 nm.

UV-Visible Spectroscopy.

The UV-Visible spectra were recorded for nanotube-DOX and nanotube-FITC conjugate on a Shimadzu UV-1800 UV-Vis spectrophotometer with slit width of 1 nm using a quartz cuvette having a path length of 1 cm with a wavelength range of 200-800 nm.

Dynamic Light Scattering (DLS).

The Nano tubular size distribution was measured on Zetasizer (Malvern, Southborough, Mass., USA). The changes in the surface charges of free nanotube and nanotube-DOX were probed via the zeta potential using Nano ZS (Malvern, Southborough, Mass., USA).

Atomic Force Microscopy (AFM).

AFM experiments were performed on a NX-10 AFM (Park systems) system in the non-contact mode. The protein sample was dropped onto freshly cleaved mica and incubated for 20 min. After rinsing with Milli-Q water two times, the sample was dried in a desiccator. The Al back-coated Si probe (ACTA, AppNano Inc, USA) had a resonance frequency of 300 kHz and nominal spring constant of 40 Nm⁻¹. The tip radius was <10 nm. Images were obtained at a scan rate of 1 Hz.

Cell Culture.

HeLa (Cervical cancer cell line), MDA-MB-231 (Breast cancer cell line) and HaCaT (Human keratinocytes) cells were maintained in growth medium consisting of Dulbecco's Modified Eagle's Medium (DMEM, Gibco, India) with high glucose, supplemented with 10% FBS and 1% penicillin-streptomycin. The cells were grown in 75 mm² flasks (BD Falcon) and passaged every three days. All culture reagents were purchased from Thermofisher scientific. The serum free media was used to eliminate any confounding effects from serum adsorption to the nanotubes. Within the time-period (5 h) of the experiment, no adverse cellular responses were observed from serum deprivation or serum shock after a transfer from serum containing growth media.

Cytotoxicity Assay.

Cytotoxicity of nanotube, DOX loaded nanotube and free DOX were assessed in HeLa and MDAMB231 using the MTT assay. About 5×10³ cells were seeded per well of a 96-well plate and allowed to attach during incubation for 24 h in a humidified incubator at 37° C. Cells were treated with different concentrations of nanotubes, free DOX or DOX loaded nanotubes ranging from 0.2 to 6.4 μg/ml. Four hours before termination of the assay, MTT was added. Mitochondria of viable cells oxidize MTT to produce purple Formazan crystals. Formazan crystals were dissolved in 100 μl of DMSO and incubated for 15 min with shaking at RT, protected from light. The absorbance of each well was measured at 570 nm using a microplate reader (Synergy HT, BioTek Instrument Inc., Winooski Vt.). Absorbance was converted to the percent cell viability and IC₅₀ concentrations calculated as the concentration that caused 50% inhibition of cell growth.

Western Blot Analysis.

Cells were maintained as mentioned above in 65 mm dishes. HeLa cells were serum starved for 24 h to remove the effect of serum on cells. Cells were treated with nanotubes or full length IGFBP-2 (1-289) for 1 h in serum-free DMEM. An additional set of cells was treated for 1 h with nanotubes or full length IGFBP-2 without the integrin pathway inhibitor (RGDS) under the same conditions. For control treatments, cells were pretreated with 10 mM RGDS peptide for 1 h. Later, cells were washed once with PBS and lysed in detergent lysis buffer (150 mM NaCl (Sigma Aldrich, USA), 0.1% SDS (Calbiochem, Germany), 0.5% NP40 (Amresco, USA), 1 mM EDTA (pH 8) and protease inhibitor cocktail, PIC (Calbiochem) with gentle vortexing at 4° C. for 20 min and centrifuged at 10,000×g for 10 min. The soluble fraction was separated and, protein estimation was done by Bradford assay. 30 μg of total protein was dissolved in SDS sample buffer (240 mM Tris-HCl (pH 6.8), 8% (w/v) SDS, 0.1% bromophenol blue and 40% (v/v) glycerol), heated at 96° C. for 10 min and centrifuged briefly. Samples were resolved on 12.5% SDS-polyacrylamide gels in Tris-glycine electrophoresis buffer (pH 8.3) containing 25 mM Tris base, 250 mM glycine and 0.1% SDS at 100 Volts for 2 h. Proteins were then transferred from the gel onto a PVDF membrane (Immobilon-P, Millipore) in 25 mM Tris base, 250 mM glycine and 20% methanol using a BioRad transfer apparatus (BioRad Laboratories, USA). Membranes were blocked with 5% non-fat dry milk solution prepared in Tris buffered-saline, pH 7.4 containing 0.1% Tween 20 (TBST) for 1 h at room temperature and then probed with the respective primary antibody at their prescribed dilutions in 2% BSA in TBST (TBT) overnight at 4° C. The following antibodies were used for Western blotting: Phospho FAK Tyr 397 (#3283, Cell signaling), Total FAK (#3285, Cell signaling) and β-Actin (# A5441, Sigma-Aldrich, USA). After primary antibody incubation, membranes were washed three times (15 min each) with TBST and incubated with HRP-conjugated secondary antibodies (Santa Cruz, USA) for 1 h at room temperature followed by washing three times (15 min each) with TBST. Enhanced chemiluminiscence substrate (#786-00, femto LUCENT™ PLUS-HRP, G-biosciences) or SuperSignal West Femto substrate (Pierce Protein Research Products, Thermo Scientific, USA) was added followed by exposing the membrane to X-ray film, development and fixation. Developer and fixer were purchased from Eastman Kodak Company, USA.

Confocal Image Acquisition and Analysis.

For microscopic analysis, cells were grown on sterile coverslips with all treatments performed in DMEM without serum for 1 h or 4 h. Treatment was terminated by removing medium and cells washing cells twice with PBS followed by nuclear staining with DAPI (4′,6-diamidino-2-phenylindole) for 10 min at room temperature. Cells were then washed with DPBS and excess buffer was removed. Coverslips were then treated with Antifade (Thermo Fisher Scientific India Private Ltd.) and sealed to a sterile glass slide and analyzed by confocal microscopy (LSM 510 Meta equipped with an Airyscan module, Zeiss, GmbH, Germany).

FACS Analysis.

Fluorescence-activated cell sorting (FACS) was used to quantify the cellular uptake of free Doxorubicin and that released from the Nanotube-DOX conjugates exposed to HeLa and HaCaT cells. The cells were treated with free DOX and Nanotube-DOX for 0.5, 1, 2 and 4 h using concentrations corresponding to their IC₅₀ values. The cells were washed with DPBS to remove the unassimilated DOX and then trypsinized. The cell suspension was then centrifuged, and the pellet was re-suspended in 300 μl of DPBS with 0.1% of FBS. The DOX uptake was estimated by quantifying the cell populations using FACS employing Cell Quest software (using a FACSCalibur instrument, BD Biosciences, San Jose Calif.).

Statistical Analysis.

Statistical analysis was performed using two-way ANOVA test. All results with p-values below 0.05 were considered as statistically significant.

### Example 2—Nanotube Self-Assembly

Monitoring the Self-Assembly of the Nanotubes.

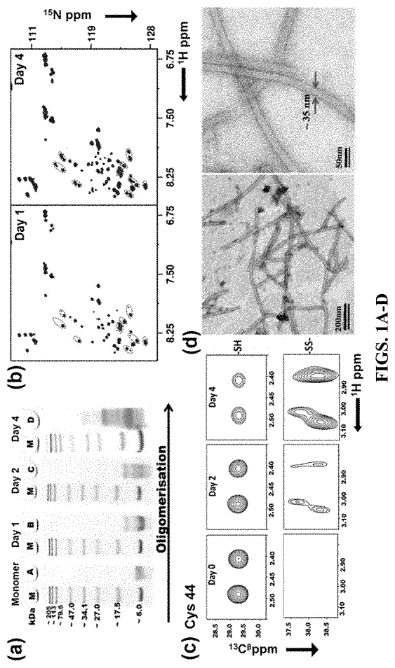

The self-assembly of hIGFBP-2₂₄₉₋₂₈₉ (R281C) was initiated at 298 K in 0.8 mM of the protein dissolved in 50 mM Na-phosphate buffer (pH 6) containing 50 mM NaCl, in the absence of any reducing agent. The oligomerization was tracked in a time-dependent manner using, in parallel, Sodium Dodecyl Sulfate-Polyacrylamide Gel Electrophoresis (SDS-PAGE) (FIG. 1a) and two-dimensional (2D) nuclear magnetic resonance (NMR) spectroscopy (FIGS. 1b and 1c). The observations were made for the total time required to form mature nanotubes i.e. 8 days at room temperature. However, emphasis was on initial time points to characterize intermediates, if any. The presence of oligomeric species was monitored by collecting fractions on Day 1, Day 2 and Day 4 and storing them at −20° C., which helped in arresting the oligomerization at different time points. The SDS-PAGE profile shown in FIG. 1a depicts the progress of oligomerization during self-assembly at initial stages at room temperature and reveals the appearance of a dimer on day 1, which becomes relatively more populated on day 2 and higher order multimeric species are visible on day 4.

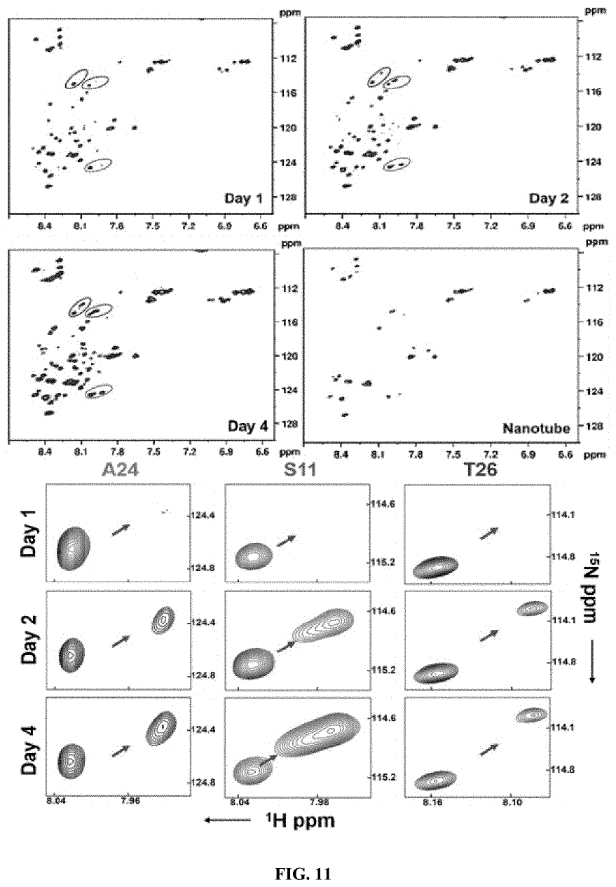

In parallel with SDS-PAGE, at the same time points, the appearance of new molecular species in solution was monitored with the help of 2D [¹⁵N, ¹H] heteronuclear single quantum correlation (HSQC) (FIG. 1b), where new spectral signatures were observed. The intensity profiles of a few resonances depicting the decrease in the signal of the monomer and appearance of new resonances as a function of time is shown in FIG. 11. The low spectral dispersion in the 2D [¹⁵N, ¹H] spectrum indicates that the polypeptide fragment is largely unstructured (FIG. 1b), which was also verified in earlier studies (Swain et al., 2010).

The self-assembly of the polypeptide fragment into nanotubular structures is governed by intermolecular disulfide formation as described previously (Swain et al., 2010). Therefore, the oxidation of the thiol groups of cysteines in the polypeptide was examined as the oligomerization progressed using a 2D [¹³C, ¹H] HSQC NMR experiment. FIG. 1c shows an expanded portion of the 2D [¹³C, ¹H] HSQC spectrum depicting the state of oxidation of one of the three cysteines, Cys44, that is, the conversion of thiol (—SH) to disulfide (—SS—) due to oxidation. During the course of self-assembly, a significant growth in the cross peak intensity of the resonance corresponding to the disulfide state is seen with the appearance and growth of the peak at ˜38 ppm, which is a characteristic chemical shift signature of the ¹³C^{β} of cysteine involved in a disulfide bond (Atreya et al., 2000). At the same time, a reduction in the intensity of thiol peaks at ˜29 ppm is observed, which is the ¹³C^{β} chemical shift for the reduced thiol group (—SH) of cysteine (Atreya et al., 2000). The other two cysteines (cys12 and cys33) were also observed to undergo a similar oxidation profile.

After completion, the nanotubes formed were observed using transmission electron microscopy (TEM). The TEM images show the hollow tubular forms (FIG. 1d). The images revealed an outer diameter of ˜35 nm and an inner diameter of ˜25 nm.

Stability of Nanotubes.

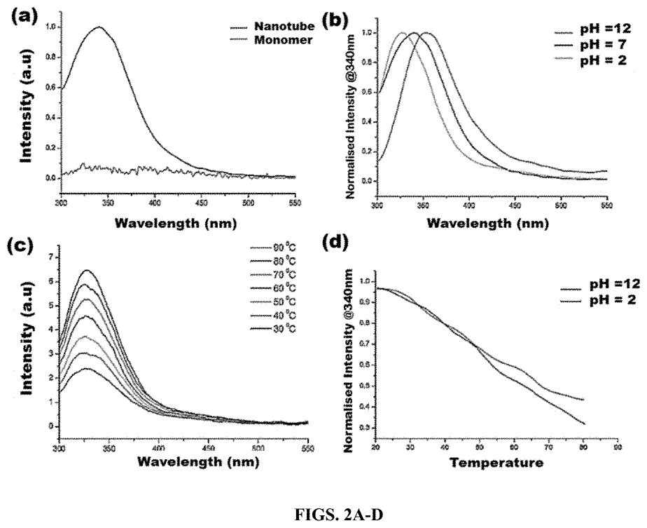

Fluorescence emission spectra were recorded at various temperatures and pH to probe the stability of nanotubes. The nanotubes have higher intrinsic tyrosine fluorescence compared to the monomer as shown in FIG. 2a, presumably due to the conformational changes in the FY dipeptide motif in the polypeptide chain (Swain et al., 2010). First, samples containing the nanotubes were prepared in three different buffers having a pH of 2, 7 and 12. Fluorescence spectra were acquired for these samples. As shown in FIG. 2b, the nanotubes were found to be stable at all the three pH. Under acidic conditions (pH 2) the emission (λₘₐₓ) shifted to a lower wavelength (blue shift) and in basic condition (pH 12) the λ max shows a red shift compared to the neutral pH (FIG. 2b). These shifts could be due to changes in the local environment of the tyrosine. Next, fluorescence spectra were acquired for nanotubes at pH 7 at different temperatures (FIG. 2c). With increases in temperature the intensity of the fluorescence emission spectrum decreases, implying quenching of fluorescence with a rise in temperature. However, even at high temperatures (90° C.) the nanotubes are intact, exhibiting significant fluorescence (tyrosine excitation 280 nm) with λₘₐₓ at 340 nm (FIG. 2c). The variable temperature studies were subsequently performed at pH 2 and pH 12. FIG. 2d shows the plot of the fluorescence emission at λₘₐₓ 340 nm for the nanotubes at pH 2 and pH 12.

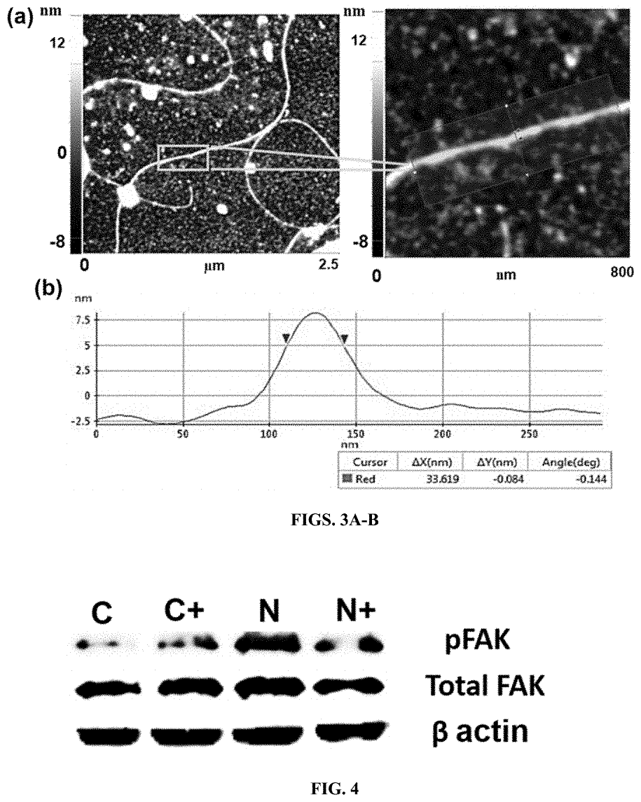

Investigation using atomic force microscopy (AFM) of the mature form of nanotubes (˜9-months old) revealed that the morphological characteristics of nanotubes were intact, showing a line profile with diameter of ˜30 nm (FIG. 3). Taken together, this implies that these protein nanotubes possess remarkable stability and can be used for various nano-platforms for biomedical applications.

### Example 3—Cellular Interactions of Nanotubes

Effect of Nanotube on Integrin Signaling.

To probe the accessibility and conformational compatibility of the RGD sites on the nanotube for binding integrins on the cell surface, integrin-induced phosphorylation of FAK at tyrosine 397 (pFAK^{Tyr-397}) was used as a measure of integrin engagement and signaling. Interaction of the RGD motif present on the nanotubes with cells via integrin binding leads to an induction of intracellular phosphorylation of FAK. Results shown in FIG. 4a indicate phosphorylation of FAK upon the treatment of HeLa cells with nanotubes (500 ng/ml) for 1 h. Pre-treatment with RGDS peptide, an integrin inhibitor, demonstrates the specificity of this interaction. In the absence of inhibitor, the intensity of pFAK was higher compared to control cells. In the presence of the inhibitor, pFAK induction by nanotubes is compromised while control cells do not exhibit significant change upon inhibitor treatment. This observation suggests that nanotubes interact with integrins at the cell surface.

Nanotube-FITC Conjugate for Imaging Cancer Cells.

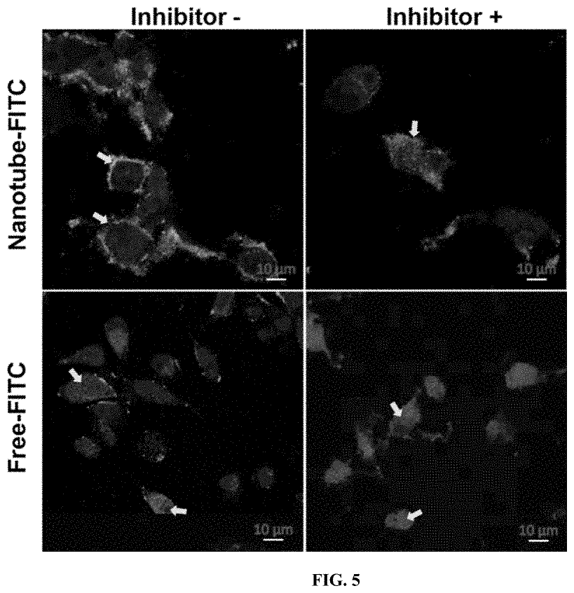

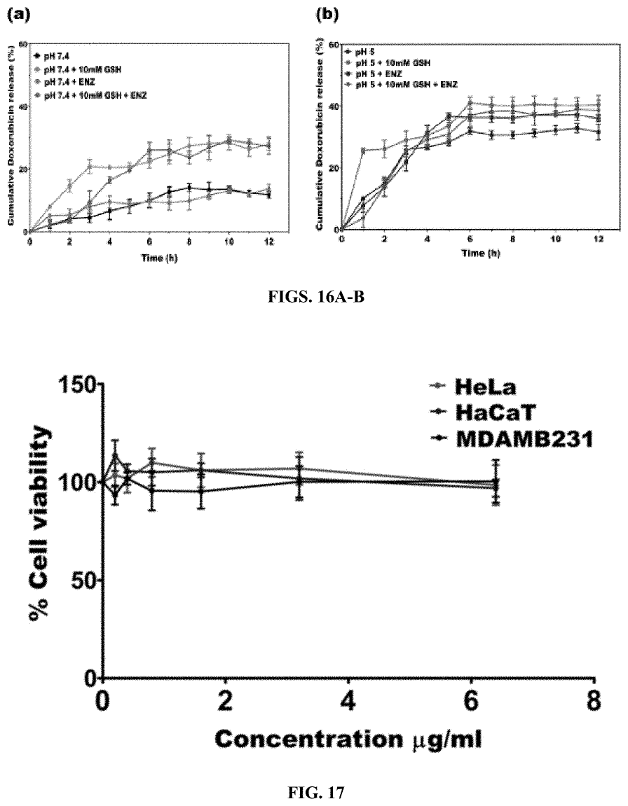

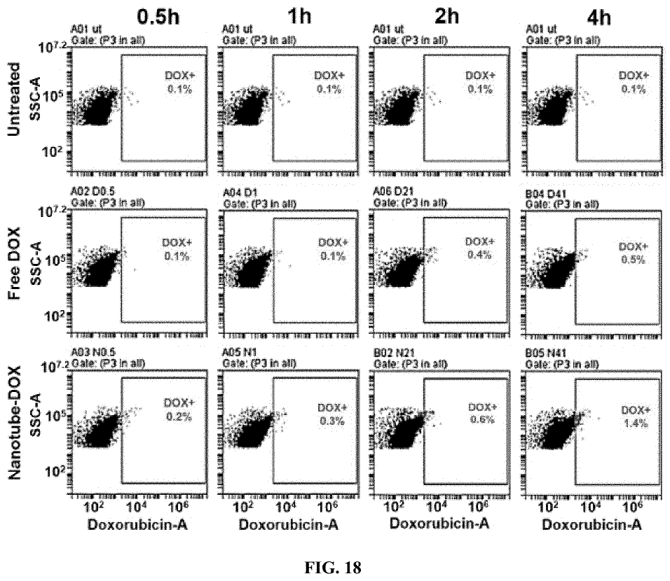

For imaging cancer cells, nanotubes were conjugated with a fluorescent dye, fluorescein isothiocyanate (FITC). FITC is a protein labeling reagent useful for fluorescence imaging (Zheng et al., 2014). FITC was covalently tagged through the side chain epsilon amine of lysine present in the polypeptide (FIG. 16). HeLa cells were treated with 300 ng/ml of nanotube-FITC conjugate for 4 h in serum free medium. FIG. 5 shows the images, which confirm the interaction of nanotubes with the cell surface and also implies that for the time points studied, the nanotube conjugate is located on the cell surface; this suggests that nanotubes can be utilized for release of a drug into the intracellular environment. The cellular uptake of nanotube-FITC by HeLa cells was visualized in absence and presence of the integrin inhibitor peptide: RGDS. This was done in order to verify that the nanotube conjugate interacts with the cell surface through integrins. Free FITC was used as control for cellular interaction via rapid diffusion in HeLa cells. Upon treatment of cells with nanotube-FITC conjugate at a concentration of 360 ng/ml, no significant internalization of nanotube-FITC conjugate is observed. The nanotube-FITC is mainly distributed over the cell surface. To evaluate competitive displacement of the nanotubes by the RGD inhibitor, cells were treated for 1 h with 25 μg/ml of inhibitor after 4 h of nanotube-FITC incubation with cells. FIG. 5 shows prominent loss of nanotube-FITC intensity after treatment with the inhibitor. On the other hand, free FITC does not show a significant decrease in its internalization profile in the presence or absence of the inhibitor. This demonstrates the specificity of nanotube-FITC interactions with integrin receptors on the cell surface and underscores the potential of the nanotubes in conjugation with fluorescent dyes as a potential tool for cancer cell imaging.

Nanotube as a Drug Carrier: Nanotube-DOX.

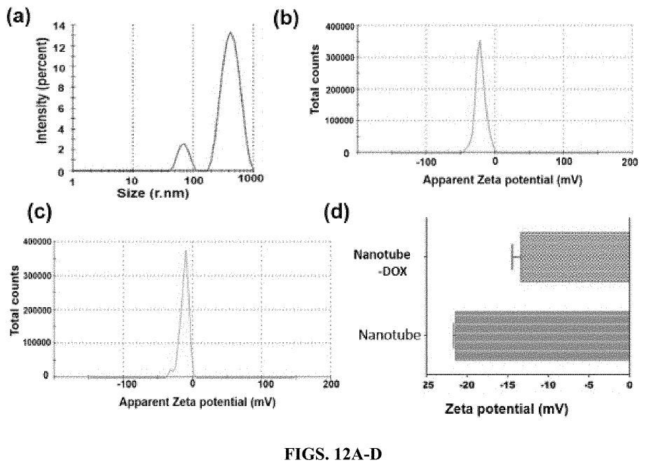

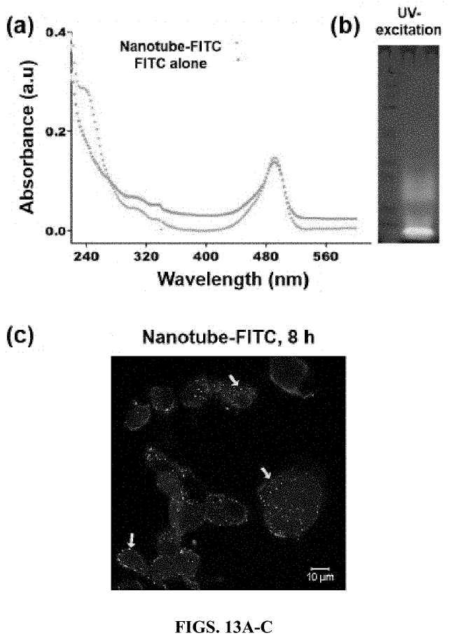

For targeted drug delivery, the presence of multiple RGD units on the nanotubes was employed. Doxorubicin (DOX) was used to measure drug release from nanotubes under pH and redox stimuli. DOX is known to exhibit its antitumor ability by interacting with the cellular DNA in nuclei of cancer cells leading to inhibition of transcription (Luo et al., 2017). The hIGFBP-2₂₄₉₋₂₈₉ nanotube constituting a combination of polar and non-polar amino acids along the frame of tubular surfaces makes it an efficient nanocarrier for DOX molecules via electrostatic and hydrophobic interactions. DOX has two possibilities for getting entrapped, either on the exterior of the tube or in the interior (hollow space) of the tube. Zeta potential was used as a measure to probe the electrostatic interaction between DOX and the nanotubes and is shown in FIGS. 12B-12D. The zeta potential decreases from −21.4 mV to −12.2 mV for the DOX loaded nanotube, which can be attributed to a rise from the electrostatic attachment of Doxorubicin to the nanotube. An efficient loading is evident from the agarose gel profile of nanotube-DOX and characteristic spectral signature seen from UV-Vis spectra in the region 490-500 nm after loading the drug (FIGS. 13a and 13c). Nanotube-DOX was drop casted on a glass surface and allowed to dry, followed by diffusion limited spreading and confocal imaging, which confirmed loading of the drug in the interior and exterior of the nanotubes (FIG. 13b).

The efficiency of drug loading was estimated by UV-Vis spectroscopy. The drug loading content (LC) and drug encapsulation efficiency (EE) were estimated, which are important parameters that characterize the performance of anti-cancer drug delivery system (Biabanikhankandani et al., 2016). The nanotube-DOX had a LC of 11% and EE of 55%.

DOX Release Profile In Vitro.

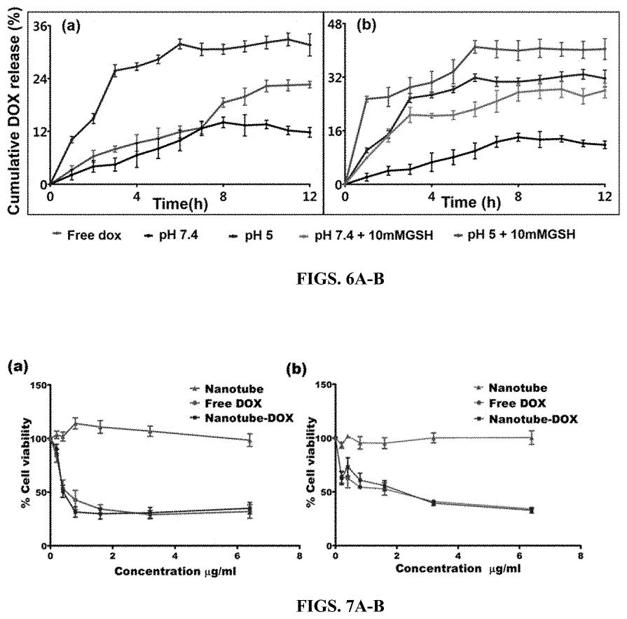

In vitro drug release profiles were obtained at different time points under simulated physiological: pH 7.4, 37° C. and tumor tissue/endosomal conditions: pH 5, 37° C. using DOX absorbance intensity as a measure (Kobayashi et al., 2014). At physiological pH, DOX release profile of the nanotube-DOX conjugate was compared with free DOX. The release of free DOX into the medium was found to be faster than that of DOX loaded on the nanotube (FIG. 6a). This can be attributed to the association of DOX with the nanotube surface via physical adsorption, which holds the DOX from rapid release compared to free DOX. Notably, significant differences are seen in the cumulative release rate of DOX from nanotube-DOX under different pH stimuli (FIG. 6a). The release is much faster and effective at pH 5 due to protonation of loaded DOX on the nanotube, which results in its weaker binding to the nanotube.

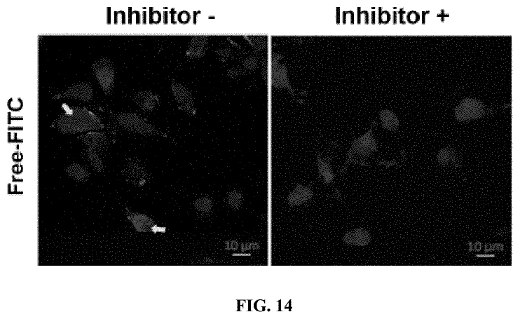

To examine the redox responsiveness of DOX released from nanotube-DOX, 10 mM GSH was added into the release medium. Despite the addition of GSH, the stimulated in vitro pH conditions were maintained. This is due to the phosphate salt present in the medium (0.05 M) which is tolerant to the addition of GSH and does not result in the change the pH (Biabanikhankandani et al., 2016). Notably, upon addition of GSH, the release rate of DOX increases (FIG. 6b), which can be attributed to the reduction of disulfide bonds under redox conditions, thereby breaking the nanotubes (Swain et al., 2010). After 6 h, the cumulative drug released at pH 7.4 is 2-fold higher in presence of GSH, whereas at pH 5, in presence of GSH, the release is ˜1.5-fold higher compared to that without GSH (FIG. 6b). The effect of a nonspecific enzyme (trypsin) on the release profiles of DOX from nanotube-DOX under physiological pH and pH 5 with stimulated redox condition was also examined (FIG. 14). These observations indicate the presence of the non-specific enzyme does not significantly affect the release rate of DOX from the nanotube-DOX.

Cytotoxicity.

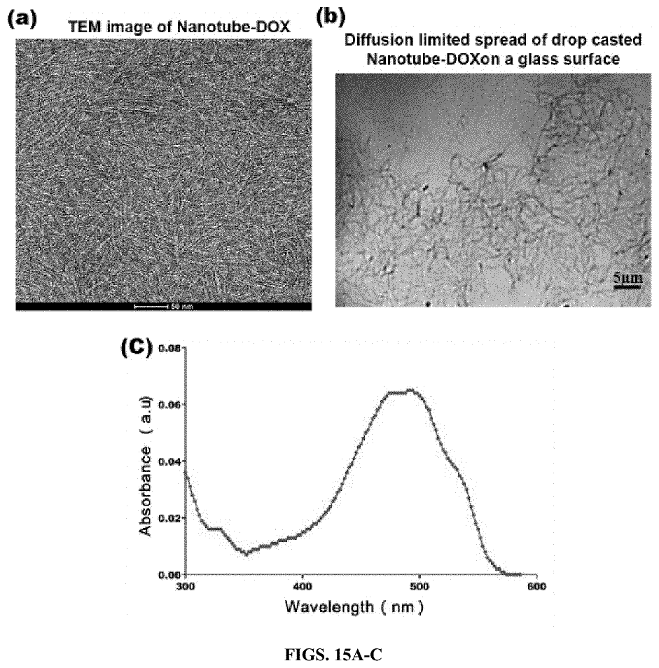

In vitro cytotoxicity of the nanotubes was measured for model cell lines: HeLa, MDAMB231 (FIG. 7) and the non-tumorigenic keratinocytes (HaCaT cells; (FIG. 15). Cytotoxicity evaluation was done using MTT assay. First, the biocompatibility of the nanotube alone in both the cell lines was examined. As shown in FIG. 7, hIGFBP2₂₄₉₋₂₈₉ nanotube treatment did not cause any appreciable cytotoxicity after 48 h, suggesting biocompatibility with these cell lines. On the other hand, treatment with 5 μg/ml of DOX for 48 h could kill almost 70% of MDAMB231 and HeLa cells. In Hela cells, the IC₅₀ for nanotube-DOX is 0.4 μg/ml and for free DOX is 0.45 μg/ml. In the case of MDAMB231, the IC₅₀ was 2 μg/ml for the Nanotube-DOX and 2.3 μg/ml for free DOX. This implies that the nanotube-DOX does not affect the efficiency of free DOX encapsulated within, and that the nanotube is delivering the drug with minimal cytotoxicity. The nanotube-DOX system did not show any cytotoxicity to the non-tumorigenic cells (FIG. 15).

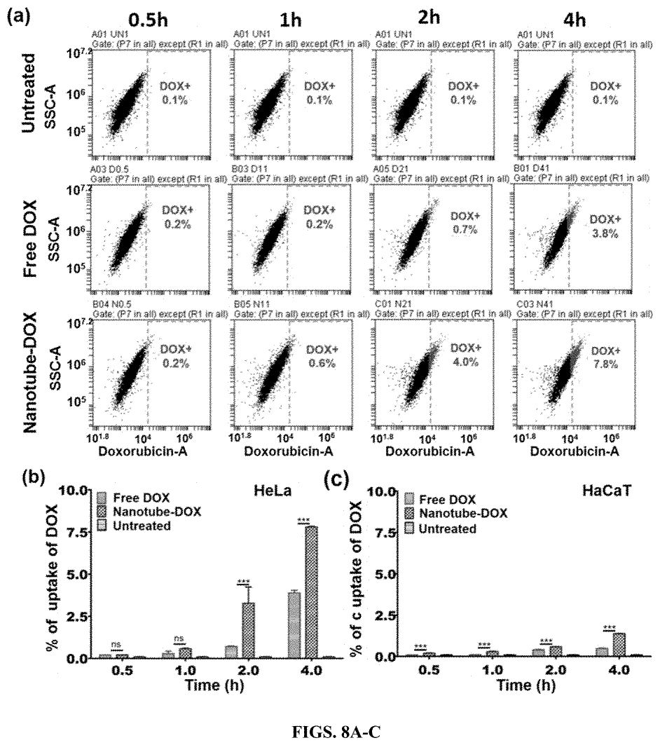

To quantify the cellular uptake of DOX loaded onto nanotubes, fluorescence assisted cell sorting (FACS) and confocal imaging were used. The investigation was done in HeLa and normal keratinocytes (HaCaT). The intracellular drug release characteristics of nanotube-DOX and free DOX were compared at concentration of 0.4 μg/ml for each. The uptake of DOX was quantified in terms of percentage population. Early time points: 0.5 h, 1 h, 2 h and 4 h were chosen to differentiate the targeting and release kinetics of DOX loaded onto the nanotube when compared to free DOX. As shown in FIGS. 8a and 8b, significant uptake of DOX is observed from 2 h onwards in HeLa cells in the nanotube-DOX system (FIG. 8b) and the uptake of DOX is minimal in the normal cells at 2 h. From 2 h to 4 h, increase in Dox uptake for free DOX is more than 5 fold, whereas it is ˜2 fold for nanotube-DOX. This indicates sustained release of DOX from the nanotube.

Visualization of Cellular Uptake of DOX.

To visualize the drug uptake in HeLa cells, confocal images were acquired after treating the cells with free DOX and DOX loaded nanotubes at the same time points using 0.4 μg/ml each. FIG. 9 depicts the internalization of drug upon treatment with free DOX and nanotube-DOX at two-time points 1 h and 4 h. In the case of free DOX, major accumulation is seen within nuclei which may be attributed to the rapid diffusion of drug molecules through the cell. Whereas the Nanotube-DOX exhibits significant accumulation of drug at the cell membrane and cytoplasm, resulting from nanotube interaction with integrins at the cell surface.

Nano drug delivery systems are considered as one of the most prospective platforms for cancer therapy because of their important physicochemical properties, including increased drug accumulation in solid tumors by the enhanced permeability and retention (EPR) effect and reduced side effects (Kobayashi et al., 2014). In recent years, RGD based targeted delivery systems have come into focus due to their specificity and efficacy (Montet et al., 2006; Welsh et al., 2013; Sun et al., 2016; Babu et al., 2017; Wang et al., 2016; Kim et al., 2009; Saraf et al., 2015). For instance, multivalent RGD based peptide nanoparticles have been proposed (Montet et al., 2006). A self-assembled multivalent RGD-peptide array has been demonstrated for integrin binding (Welsh et al., 2013). Functional self assembling RGD containing peptide nanofiber hydrogel was designed for nerve generation (Sun et al., 2016). Disulfide based multifunctional RGD containing conjugate was introduced for targeted theranostic drug delivery (Lee et al., 2015). Disulphide connectivity is known to be stable in blood pool but is efficiently cleaved by cellular thiols, including Glutathione and Thioredoxin (Lee et al., 2015).

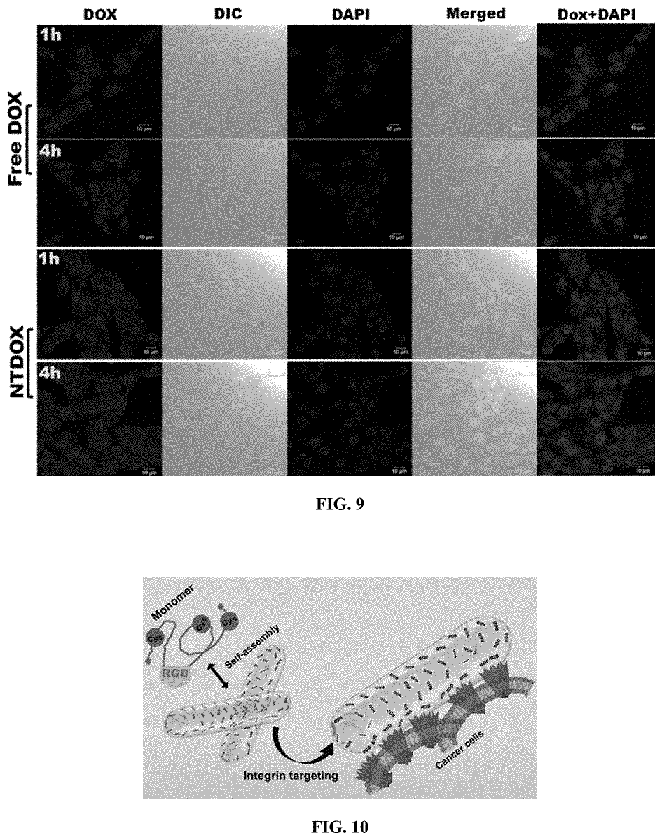

The present study introduces the first example of a water soluble self-assembling nanotube formed by a polypeptide fragment from a human protein, which can effectively target cancer cells, providing a platform for imaging of cells, and design and delivery of suitable therapeutics to the cells. The nanotube is formed via redox controlled self-assembly and the array of multiple RGD motifs present on the nanotube makes it suitable for targeting cancer cells via the integrin pathway as verified by the induction of integrin mediated pFAK signaling and cellular uptake in the presence and absence of an integrin inhibitor. This is depicted schematically in FIG. 10.

The polypeptide fragment is derived from a natural source i.e. polypeptide fragment of human insulin-like growth factor binding protein-2. It therefore exhibits excellent biocompatibility which is reflected in the in vitro cytotoxicity evaluation in cancer cells. The intermolecular disulfide formation drives self-assembly of the polypeptide into nanotubular forms providing a stable framework. The nanotubes are stable over a wide range of temperature and pH exhibiting protease-resistance due to Cys²⁸¹ mutation, which is known to be one of the proteolytic sites in human IGFBP2 (Mark et al., 2005). In addition, the polypeptide hIGFBP-2₂₄₉₋₂₈₉, being part of C-terminal domain of full length IGFBP-2 lacks major proteolytic sites and hence is expected to be unaffected by proteases (Mark et al., 2005; Soh et al., 2014).

Notably, no additional modification such PEGylation is needed, which is commonly used for providing biocompatibility to various nanoparticles (Miller et al., 2010) and physical rigidity to the nanoplatforms (Huang et al., 2017). Our experiments reveal the potential of this system for targeted therapeutics that includes drug delivery and imaging. In vitro drug release profiles confirm its ability to effectively deliver the cargo at target site. Disulfide connectivity makes to redox responsive to the cellular GSH and lysosomal environment, which again renders the nanoplatform as a biocompatible redox responsive system apt for targeted therapy.

In summary evidence is provided herein for RGD based natural polypeptide nanotubes as effective delivery system for imaging and treatment with cytotoxic drugs. The targeted delivery has the potential to minimize the ill effects of the cytotoxic drugs on normal surrounding cells thus alleviating the side effects during the treatment of cancer.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Alibolandi, M.; Abnous, K.; Sadeghi, F.; Hosseinkhani, H.; Ramezani, M.; Hadizadeh, F., Folate receptor-targeted multimodal polymersomes for delivery of quantum dots and doxorubicin to breast adenocarcinoma: In vitro and in vivo evaluation. Int. J. Pharm. 2016, 500, 162-78.
2. Cuggino, J. C.; Molina, M.; Wedepohl, S.; Igarzabal, C. I. A.; Calderon, M.; Gugliotta, L. M., Responsive nanogels for application as smart carriers in endocytic pH-triggered drug delivery systems. Eur. Polym. J. 2016, 78, 14-24.
3. Wei, T.; Chen, C.; Liu, J.; Liu, C.; Posocco, P.; Liu, X.; Cheng, Q.; Huo, S.; Liang, Z.; Fermeglia, M.; Pricl, S.; Liang, X.-J.; Rocchi, P.; Peng, L., Anticancer drug nanomicelles formed by self-assembling amphiphilic dendrimer to combat cancer drug resistance. Proc. Natl. Acad. Sci. U.S.A 2015, 112, 2978-83.
4. Tomitaka, A.; Arami, H.; Huang, Z.; Raymond, A.; Rodriguez, E.; Cai, Y.; Febo, M.; Takemura, Y.; Nair, M., Hybrid magneto-plasmonic liposomes for multimodal image-guided and brain-targeted HIV treatment. Nanoscale 2018, 10, 184-94.
5. Poshteh Shirani, M.; Rezaei, B.; Khayamian, T.; Dinari, M.; Karami, K.; Mehri-Lighvan, Z.; Hosseini Shamili, F.; Ramazani, M.; Alibolandi, M., Folate receptor-targeted multimodal fluorescence mesosilica nanoparticles for imaging, delivery palladium complex and in vitro G-quadruplex DNA interaction. J. Biomol. Struct. Dyn. 2017, 35, 1-14.
6. Wang, M.; Thanou, M., Targeting nanoparticles to cancer. Pharmacol. Res. 2010, 62, 90-99.
7. Bellis, S. L., Advantages of RGD peptides for directing cell association with biomaterials. Biomaterials 2011, 32, 4205-10.
8. Arnaout, M. A.; Mahalingam B Fau-Xiong, J. P.; Xiong, J. P., Integrin structure, allostery, and bidirectional signaling. Annu. Rev. Cell. Dev. Biol. 2005, 21, 381-410.
9. Desgrosellier, J. S.; Cheresh, D. A., Integrins in cancer: biological implications and therapeutic opportunities. Nat. Rev. Cancer 2010, 10, 9-22.
10. Hynes, R. O., Integrins: Bidirectional, Allosteric Signaling Machines. Cell 2002, 110, 673-87.
11. Swain, M.; Thirupathi, R.; Krishnarjuna, B.; Eaton, E. M.; Kibbey, M. M.; Rosenzweig, S. A.; Atreya, H. S., Spontaneous and reversible self-assembly of a polypeptide fragment of insulin-like growth factor binding protein-2 into fluorescent nanotubular structures. Chem. Commun. 2010, 46, 216-18.
12. Atreya, H. S.; Sahu Sc Fau-Chary, K. V.; Chary Kv Fau-Govil, G.; Govil, G., A tracked approach for automated NMR assignments in proteins (TATAPRO). J. Biomol. NMR 2000, 17, 125-36.
13. Zheng, Y.; Ji, S.; Czerwinski, A.; Valenzuela, F.; Pennington, M.; Liu, S., FITC-Conjugated Cyclic RGD Peptides as Fluorescent Probes for Staining Integrin αvβ3/αvβ5 in Tumor Tissues. Bioconjug. Chem. 2014, 25, 1925-41.
14. Luo, D.; Goel, S.; Liu, H.-J.; Carter, K. A.; Jiang, D.; Geng, J.; Kutyreff, C. J.; Engle, J. W.; Huang, W.-C.; Shao, S.; Fang, C.; Cai, W.; Lovell, J. F., Intrabilayer 64Cu Labeling of Photoactivatable, Doxorubicin-Loaded Stealth Liposomes. ACS Nano 2017, 11, 12482-91.
15. Biabanikhankandani, R.; Alitheen, N. B.; Ho, K. L.; Tan, W. S., pH-responsive Virus-like Nanoparticles with Enhanced Tumour-targeting Ligands for Cancer Drug Delivery. Sci. Rep. 2016, 6, 37891-904.
16. Kobayashi, H.; Watanabe, R.; Choyke, P. L., Improving Conventional Enhanced Permeability and Retention (EPR) Effects; What Is the Appropriate Target? Theranostics 2014, 4, 81-89.
17. Montet, X.; Funovics, M.; Montet-Abou, K.; Weissleder, R.; Josephson, L., Multivalent Effects of RGD Peptides Obtained by Nanoparticle Display. J. Med. Chem. 2006, 49, 6087-93.
18. Welsh, D. J.; Posocco, P.; Pricl, S.; Smith, D. K., Self-assembled multivalent RGD-peptide arrays—morphological control and integrin binding. Org. Biomol. Chem. 2013, 11, 3177-86.
19. Sun, Y.; Li, W.; Wu, X.; Zhang, N.; Zhang, Y.; Ouyang, S.; Song, X.; Fang, X.; Seeram, R.; Xue, W.; He, L.; Wu, W., Functional Self-Assembling Peptide Nanofiber Hydrogels Designed for Nerve Degeneration. ACS Appl. Mater. Interfaces 2016, 8, 2348-59.
20. Babu, A.; Amreddy, N.; Muralidharan, R.; Pathuri, G.; Gali, H.; Chen, A.; Zhao, Y. D.; Munshi, A.; Ramesh, R. A.-O. h. o. o., Chemodrug delivery using integrin-targeted PLGA-Chitosan nanoparticle for lung cancer therapy. Sci. Rep. 2017, 7, 14674-90.
21. Wang, Z.; Dong, L.; Han, L.; Wang, K.; Lu, X.; Fang, L.; Qu, S.; Chan, C. W., Self-assembled Biodegradable Nanoparticles and Polysaccharides as Biomimetic ECM Nanostructures for the Synergistic effect of RGD and BMP-2 on Bone Formation. Sci. Rep. 2016, 6, 25090-102.
22. Kim, J.-K.; Anderson, J.; Jun, H.-W.; Repka, M. A.; Jo, S., Self-Assembling Peptide Amphiphile-Based Nanofiber Gel for Bioresponsive Cisplatin Delivery. Mol. Pharm. 2009, 6, 978-85.
23. Saraf, P.; Li, X.; Wrischnik, L.; Jasti, B., In Vitro and In Vivo Efficacy of Self-Assembling RGD Peptide Amphiphiles for Targeted Delivery of Paclitaxel. Pharm. Res. 2015, 32, 3087-101.
24. Lee, M. H.; Sessler, J. L.; Kim, J. S., Disulfide-Based Multifunctional Conjugates for Targeted Theranostic Drug Delivery. Acc. Chem. Res. 2015, 48, 2935-46.
25. Mark, S.; Kübler, B.; Höning, S.; Oesterreicher, S.; John, H.; Braulke, T.; Forssmann, W.-G.; Ständker, L., Diversity of Human Insulin-like Growth Factor (IGF) Binding Protein-2 Fragments in Plasma: Primary Structure, IGF-Binding Properties, and Disulfide Bonding Pattern. Biochemistry 2005, 44, 3644-52.
26. Soh, C. L.; McNeil, K.; Owczarek, C. M.; Hardy, M. P.; Fabri, L. J.; Pearse, M.; Delaine, C. A.; Forbes, B. E., Exogenous administration of protease-resistant, non-matrix-binding IGFBP-2 inhibits tumour growth in a murine model of breast cancer. Br. J. Cancer 2014, 110, 2855-64.
27. Miller, J. S.; Shen, C. J.; Legant, W. R.; Baranski, J. D.; Blakely, B. L.; Chen, C. S., Bioactive hydrogels made from step-growth derived PEG-peptide macromers. Biomaterials 2010, 31, 3736-43.
28. Huang, X.; Chisholm, J.; Zhuang, J.; Xiao, Y.; Duncan, G.; Chen, X.; Suk, J. S.; Hanes, J., Protein nanocages that penetrate airway mucus and tumor tissue. Proc. Natl. Acad. Sci. U.S.A. 2017, 114, 6595-6602.
29. Kibbey, M. M.; Jameson, M. J.; Eaton, E. M.; Rosenzweig, S. A., Insulin-Like Growth Factor Binding Protein-2: Contributions of the C-Terminal Domain to Insulin-Like Growth Factor-1 Binding. Mol. Pharm. 2006, 69, 833-45.
30. Atreya, H. S.; (ed.). Isotope labeling in Biomolecular NMR 2012 (Springer, Netherlands).
31. Muchmore, D. C.; McIntosh, L. P.; Russell, C. B.; Anderson, D. E.; Dahlquist, F. W., Expression and nitrogen-15 labeling of proteins for proton and nitrogen-15 nuclear magnetic resonance. Methods Enzymol. 1989; 177, 44-73.
32. Lowry, 0. H.; Rosebrough, N. J.; Farr, A. L.; Randall, R. J., Protein measurement with Folin phenol reagent. J. Biol. Chem. 1951, 193, 265-75.
33. Goding, J. W., Conjugation of antibodies with fluorochromes: modifications to the standard methods. J. Immunol. Methods 1976, 13, 215-26.
34. Rinderknecht, H., A new technique for the fluorescent labelling of proteins. Experientia 1960, 16, 430-31.

1. A therapeutic composition comprising a polypeptide tubule composed of polypeptide subunits that are linked by cysteine disulfide bonds and a therapeutic molecule encapsulated in said polypeptide tubule.

2. The composition of claim 1, wherein the polypeptide subunits each have identical sequences.

3. The composition of claim 1, wherein the polypeptide subunits are 10-100 amino acids in length.

4. The composition of claim 1, wherein the polypeptide subunits comprise an RGD motif.

5. The composition of claim 4, wherein the polypeptide tubule can bind to cell surface integrin.

6. The composition of claim 1, wherein the polypeptide subunits each comprise at least 3 cysteine positions.

7. The composition of claim 1, wherein the polypeptide subunits comprise an intramolecular disulfide bond between a first and second cysteine of the same subunit and an intermolecular bond between a third cysteine and a cysteine from a different subunit.

8. The composition of claim 1, wherein the polypeptide subunits comprise an amino acid sequence at least 80% identical to CVNPNTGKLIQGAPTIRGDPECHLFYNEQQEACGVHTQRMT (SEQ ID NO: 2) or GPLGSPGIRGSCVNPNTGKLIQGAPTIRGDPECHLFYNEQQEACGVHTQRMT (SEQ ID NO: 1).

9. The composition of claim 8, wherein the polypeptide subunits comprise an amino acid sequence at least 85%, 86%, 87%, 88%, 89%, 90%, 91%, 92%, 93%, 94%, 95%, 96%, 97%, 98% or 99% identical to SEQ ID NO: 2.

10. The composition of claim 9, wherein the polypeptide subunits comprise an amino acid sequence identical to SEQ ID NO: 2.

11. The composition of claim 8, wherein the polypeptide subunits comprise an amino acid sequence at least 85%, 86%, 87%, 88%, 89%, 90%, 91%, 92%, 93%, 94%, 95%, 96%, 97%, 98% or 99% identical to SEQ ID NO: 1.

12. The composition of claim 11, wherein the polypeptide subunits comprise an amino acid sequence identical to SEQ ID NO: 1.

13. The composition of claim 1, wherein the polypeptide tubule is PEGylated.

14. The composition of claim 1, wherein the therapeutic molecule is a cytotoxic agent.

15. The composition of claim 1, wherein the therapeutic molecule is a chemotherapy, radiotherapy, gene therapy, surgery, hormonal therapy, anti-angiogenic therapy or cytokine therapy agent.

16. The composition of claim 1, wherein the therapeutic molecule is a chemotherapeutic agent.

17. The composition of claim 16, wherein the therapeutic molecule is Doxorubicin.

18. A method of treating a subject in need thereof comprising administering an effective amount of a composition in accordance with claim 1.

19. The method of claim 18, wherein the therapeutic molecule is a chemotherapeutic agent and the wherein the subject has a cancer.

20. The method of claim 19, wherein the cancer is oral cancer,
oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

21-27. (canceled)
